(19) United States
(12) Patent Application Publication
(10) Pub. No.: US 2004/0133270 A1
(43) Pub. Date: Jul. 8, 2004

(54) Drug eluting stent and methods of manufacture

(76) Inventor: Axel Grandt, Strassberg (DE)

Correspondence Address: Nicola A. Pisano, Esq., Suite 200, 11988 El Camino Real, San Diego, CA, 92130, US

(21) Appl. No.: 10/616,125

(22) Filed: Jul. 8, 2003

Related U.S. Application Data
(60) Provisional application No. 60/394,978, filed on Jul. 8, 2002.

(51) Int. Cl. A61F 2/06

(52) U.S. Cl. 623/1.42

(57) ABSTRACT

Apparatus and methods for manufacturing a drug eluting stent are provided, whereby the stent comprises at least one tube having a lumen and a multiplicity of microscopic pores disposed in a lateral surface of the tube. The tube may be manufactured into any suitable stent configuration. The lumen of the tube is configured to retain a therapeutic agent that may be eluted through the multiplicity of pores into a vessel after deployment of the stent.

### FIELD OF THE INVENTION

The present invention relates to stents, and more particularly, to a stent having a lumen and a multiplicity of microscopic pores that communicate with the lumen so that a therapeutic agent may be eluted into a vessel subsequent to deployment of the stent.

### BACKGROUND OF THE INVENTION

Balloon angioplasty, either alone or followed by stent implantation, has become a commonplace interventional alternatives to open heart surgery in those patients appropriate for such treatment. Stents are generally tubular members having a contracted state suitable for insertion into a vessel and a deployed state in which the stent is expanded to support the surrounding tissue and prevent at least local narrowing of the vessel. Several types of stents are known, including balloon expandable, self-expanding, and stents constructed from bistable springs.

One problem arising from the use of the foregoing interventional techniques, however, is that the treated vessel may restenose shortly after the interventional procedure. Restenosis is defined as the recurrence of a constriction in a blood vessel after it has been treated with apparent success, e.g., using balloon angioplasty. Clinical data suggests that there is about a 35-45% rate of restenosis in patients that undergo balloon angioplasty as the sole means of treatment for coronary artery stenoses. Where a stent is deployed subsequent to a balloon angioplasty procedure, clinical data suggests that the rate of restenosis for coronary stents still is relatively high, e.g., in a range between about 20-30%.

Therapeutic drugs have been developed that attempt to reduce restenosis rates. Such drugs, when introduced systemically, may result in undesirable side effects. Previously known methods of providing such drugs in a localized manner have involved coating the stent with a drug-laden polymer coating.

More specifically, several drug eluting stents are known in which a drug is disposed in the matrix of a bioabsorbable polymer coated on an exterior surface of the stent. The drug is gradually released into an arterial wall to prevent restenosis. Clinical data suggests that restenosis rates may be reduced to less than 10% when drug eluting stents are used. However, there is a risk of adverse reaction to the polymer matrix that may reduce the effectiveness of such drug eluting stents. Furthermore, as drug eluting stents are still an emerging technology, there is room for improvement in the design of such stents.

In view of these drawbacks of previously known stents, it would be desirable to provide a stent capable of eluting a therapeutic agent over an extended period of time subsequent to deployment of the stent. The therapeutic agent may be targeted to inhibit restenosis, or to provide some alternative therapeutic goal, e.g., to release an angiogenic agent that encourages growth of the vascular bed.

It also would be desirable to provide a drug eluting stent capable of retaining a therapeutic agent in a hollow, interior portion of the stent so that the drug may be eluted to a local region of the vessel wall in a controlled manner through pores in the stent.

It further would be desirable to provide a drug eluting stent that may provide a therapeutic agent to a vessel using a variety of known stent configurations, including, e.g., self-expandable stents, balloon expandable stents and mesh stents.

### SUMMARY OF THE INVENTION

In view of the foregoing, it is an object of the present invention to provide a stent capable of eluting a therapeutic agent over an extended period of time subsequent to deployment of the stent, e.g., to reduce the likelihood of restenosis in a vessel or to encourage revascularization.

It is another object of the present invention to provide a drug eluting stent capable of retaining a therapeutic agent in a hollow, interior portion of the stent so that the drug may be eluted to a local region of the vessel wall in a controlled manner through pores in the stent.

It is another object of the present invention to provide a drug eluting stent that may provide a therapeutic agent to a vessel using a variety of known stent configurations, including, e.g., self-expandable stents, balloon expandable stents and mesh stents.

These and other objects of the present invention are achieved by providing a drug eluting stent comprising at least one tube having a lumen and multiplicity of through-wall pores that communicate with the lumen. A therapeutic agent, e.g., antiplatelet drugs, anticoagulant drugs or gene vectors, may be inserted and retained in the lumen of the stent during manufacture. Once the stent is implanted, the therapeutic agent elutes from within the lumen via the multiplicity of pores to deliver the therapeutic agent to a vessel wall in a controlled manner over an extended period of time.

In a preferred method of manufacturing the drug eluting stent of the present invention, a hollow tube having proximal and distal ends and a lumen extending therebetween is provided. A distal opening of the tube may be plugged, e.g., by welding or crimping, and the therapeutic agent is then inserted into the lumen via the proximal end. The proximal end then is plugged to confine the therapeutic agent within the lumen. preferably, the multiplicity of pores in the stent is such that the therapeutic agent is retained in the lumen until the stent is implanted. The tube then is formed into a desired stent configuration. The above-described steps are intended to be interchangeable, e.g., the pores may be formed prior to insertion of the therapeutic agent, or the desired shape of the stent may be formed prior to insertion of the therapeutic agent into the lumen.

The multiplicity of pores may be disposed on a lateral surface of the stent spaced apart at equal or variable distances with respect to one another, and may be disposed along a longitudinal axis of the tube or spaced circumferentially about a lateral surface of the tube. Additionally, the tube may comprise at least one solid section that separates the stent into individual compartments along its length.

The drug eluting stent of the present invention may be manufactured into a number of stent configurations. In a first embodiment, a tube having at least one lumen and a therapeutic agent disposed therein comprises a shape-memory material that is configured to self-deploy to form a coil-shaped stent. The therapeutic agent is retained within the stent during delivery, and exits from the lumen into the vessel through the multiplicity of pores, over an extended period of time, after the stent is deployed in a patient's vessel.

In an alternative embodiment of the present invention, a tube having a lumen and a therapeutic agent disposed therein is deformed into a configuration having a plurality of upper peaks and lower peaks. A proximal end of the tube is affixed to a distal end of the tube to form a circumferential ring, and a plurality of circumferential rings may be affixed together end-to-end to form a stent. The stent is provided in a contracted state in which it is crimped onto a balloon catheter or contained within a delivery sheath, and the stent further retains the therapeutic agent in the lumen during delivery of the stent. After the stent is deployed, the therapeutic agent is eluted from the lumen into the vessel via the multiplicity of pores disposed in a lateral surface of the stent. Alternatively, a similar stent configuration may be formed by first forming a tube into a series of sinusoids, and then wrapping that sinusoidal pattern helically about a mandrel, as described in U.S. Pat. Nos. 5,019,090 and 5,135,536.

Further alternative configurations of the drug eluting stent of the present invention may comprise a mesh stent and a stent having plurality of unit cells having a “bistable function,” defined herein as only two configurations in which it is stable without the need for an external force to hold it in that shape. Regardless of the selected stent configuration, each embodiment comprises at least one tube having at least one lumen that retains a therapeutic agent during delivery of the stent, and a multiplicity of pores through which the agent may be eluted subsequent to implantation of the stent.

### BRIEF DESCRIPTION OF THE DRAWINGS

Further features of the invention, its nature and various advantages will be more apparent from the accompanying drawings and the following detailed description of the preferred embodiments, in which:

FIG. 1A-1D are, respectively, three side-sectional views and a side view illustrating a method for manufacturing a drug eluting stent in accordance with principles of the present invention;

FIGS. 2A-2B illustrate alternative configurations of the pores of FIG. 1D;

FIG. 3 is a side-sectional view illustrating alternative lumen configurations for a tube of the present invention;

FIG. 4 illustrates a stent provided in accordance with the principles of the present invention in a deployed state;

FIGS. 5A-5B illustrate a preferred method of using the stent of FIG. 4;

FIGS. 6A-6D are, respectively, a side sectional view and three side views illustrating a method for manufacturing an alternative stent in accordance with the present invention;

FIGS. 7A-7B illustrate a preferred method of using the stent of FIG. 6D;

FIGS. 8A-8B are schematic views of an alternative stent of the present invention in contracted and deployed states, respectively; and

FIG. 9 is a side view of a further alternative embodiment of the present invention.

### DETAILED DESCRIPTION OF THE INVENTION

The present invention relates to stents, and more particularly, to a drug eluting stent comprising at least one tube having a lumen and multiplicity of microscopic pores disposed in a lateral surface of the tube. The lumen of the tube is configured to contain a therapeutic agent that may be eluted through the pores into a vessel subsequent to deployment of the stent, for example, to reduce the risk of restenosis in the vessel.

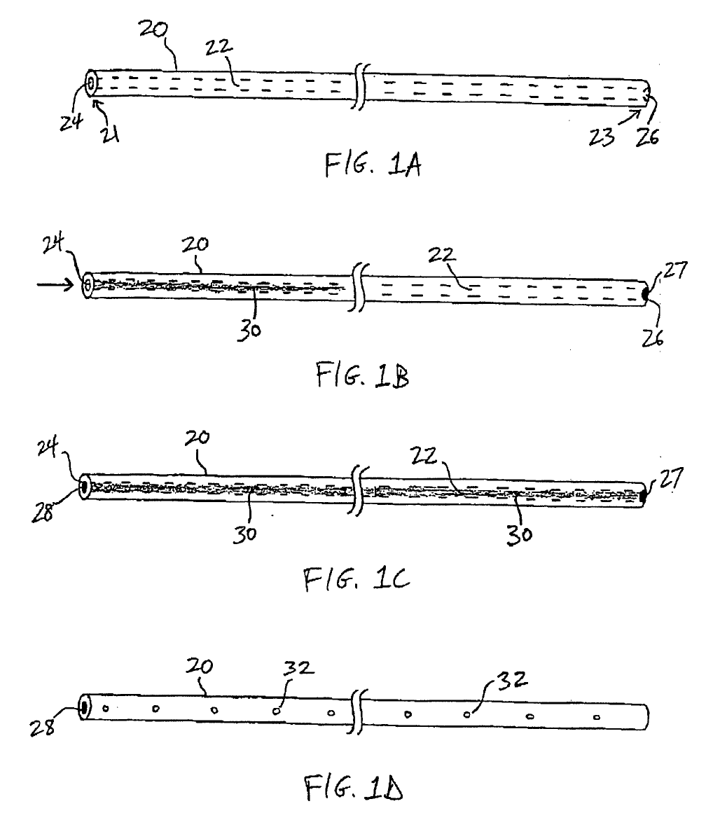

Referring now to FIGS. 1, a preferred method for manufacturing a drug eluting stent in accordance with principles of the present invention is described. In FIG. 1A, tube 20 having proximal and distal ends 21 and 23 comprises lumen 22 extending therebetween. Tube 20 preferably is manufactured using a shape-memory material, e.g., a nickel-titanium alloy, or alternatively may be manufactured from stainless steel. Tube 20 comprises proximal opening 24 and distal opening 26, each of which are in fluid communication with lumen 22, as shown in FIG. 1A.

In a preferred first step, distal opening 26 of tube 20 is plugged, e.g., using weld 27 or another appropriate means for plugging the opening. Therapeutic agent 30 then is inserted into lumen 22 through proximal opening 24, e.g., using a syringe (not shown) or other suitable means.

Therapeutic agent 30 may comprise antiplatelet drugs, anticoagulant drugs, drugs that interrupt cell replication, gene vectors, or any alternative drug or agent that is desired. Therapeutic agent 30 preferably is used in conjunction with a chemically modified bioabsorbable polymer (not shown) that slowly biodegrades over a period of time. The use of such polymer causes therapeutic agent 30 to be temporarily retained within lumen 22, then eluted through pores 32 of FIG. 1D over a period of time as a result of the exposure of the polymer to blood flow.

After the desired amount of therapeutic agent 30 has been inserted into lumen 22, proximal opening 24 preferably is plugged, e.g., using weld 28, so that therapeutic agent 30 is confined within tube 20, as shown in FIG. 1C.

Referring now to FIG. 1D, a multiplicity of pores 32 are formed in a lateral surface of tube 20 and are in fluid communication with lumen 22. Pores 32 preferably are formed using an excimer laser to achieve the preferred diameter and depth. It will be appreciated by those skilled in the art that any of the steps described in FIGS. 1B-1D may be interchanged, e.g., pores 32 may be formed prior to insertion of therapeutic agent 30.

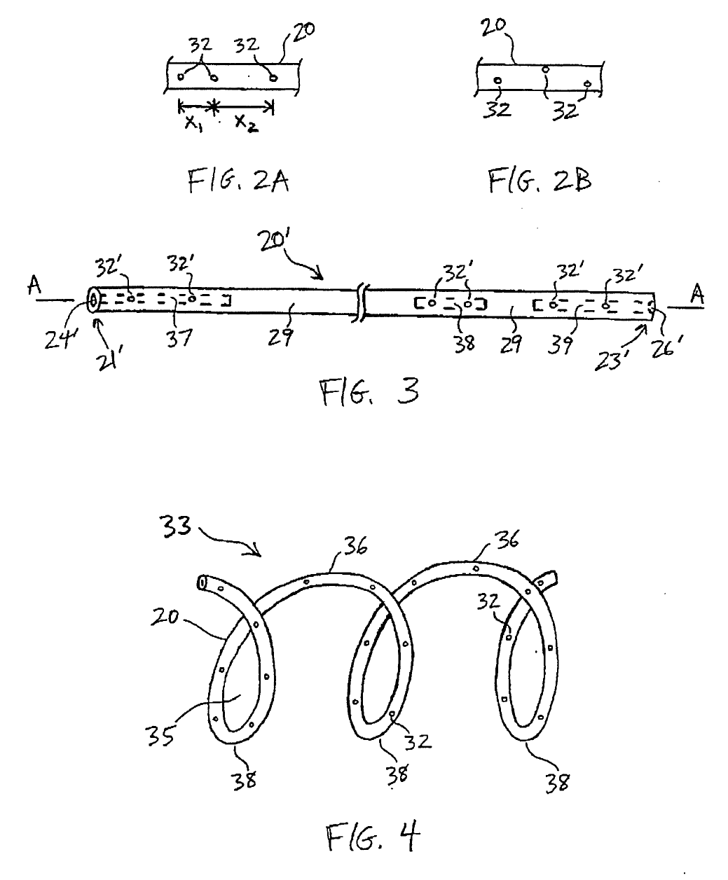

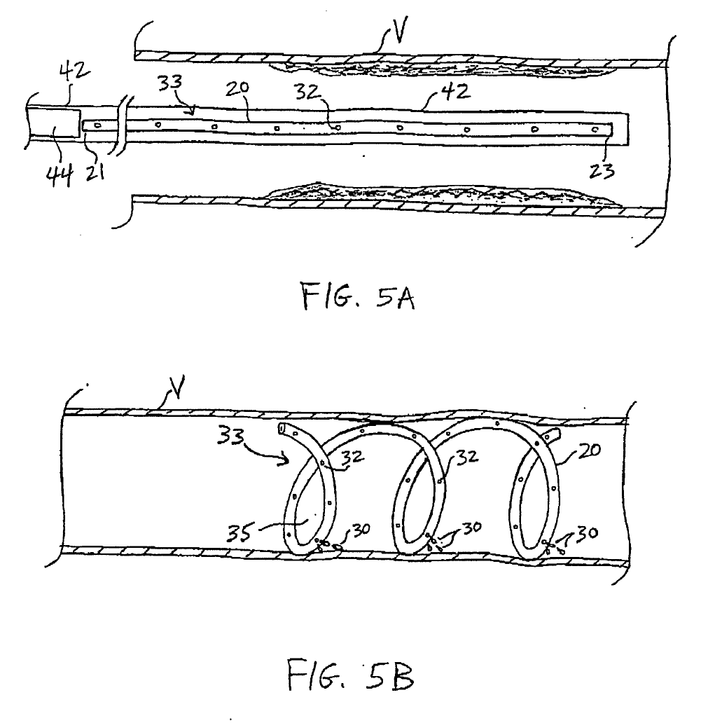

Referring now to FIGS. 2, variations in the placement of pores 32 along tube 20 are shown. In FIG. 2A, pores 32 are spaced apart at variable distances with respect to one another. For example, first and second pores may be spaced apart distance x₁ from center to center, while second and third pores may be spaced apart distance x₂ from center to center, as shown in FIG. 2A. Additionally, pores 32 may be disposed circumferentially about an exterior surface of tube 20, as depicted in FIG. 2B. Furthermore, it should be appreciated by those skilled in the art that while pores 32 are illustrated as having substantially uniform circular configurations, the pores alternatively may comprise different sizes and/or shapes, e.g., elliptical or rectangular configurations.

Referring now to FIG. 3, an alternative configuration of tube 20 of FIG. 1 is described. Partially hollow tube 20′ comprises at least one solid section 29 disposed between proximal end 21′ and distal end 23′. In this embodiment, a therapeutic agent may be inserted into partially hollow tube 20′ at selected locations along longitudinal axis A—A. For example, an agent may be inserted into proximal lumen 37 via proximal opening 24′ and may additionally be inserted into distal lumen 39 via distal opening 26′. The therapeutic agent further may be drawn into central lumen 38 via pores 32′ by applying suction to either or both ends of the hollow tube 20′. The embodiment of FIG. 3 makes it possible to provide a stent having one or more solid sections 29 while providing a therapeutic agent within desired regions along tube 20′. As will be apparent to those skilled in the art, different therapeutic agents may be disposed in different sections of tube 20′.

Referring now to FIG. 4, a first embodiment of a drug eluting stent constructed in accordance with principles of the present invention is described. In FIG. 4, coil-shaped stent 33 comprises tube 20 of FIG. 1D. As described hereinabove with respect to FIGS. 1A-1D, tube 20 comprises pores 32 disposed in a lateral surface of tube 20 and therapeutic agent 30 disposed within lumen 22 of tube 20.

In the embodiment of FIG. 4, tube 20 is configured to self-deploy to a predetermined shape comprising at least one upper peak 36 and at least one lower peak 38 that form apertures 35 through which blood may flow. Upper and lower peaks 36 and 38 maintain patency of a vessel when stent 33 is deployed.

Tube 20 preferably comprises a shape-memory material, such as a nickel-titanium alloy. During manufacture, tube 20 preferably is disposed about a mandrel in a desired deployment shape and an appropriate heat treatment is applied, as per se known in the art, to cause tube 20 to self-deploy to the predetermined shape shown in FIG. 4. Although illustrated as a helix in FIG. 4, the stent also may be formed by first forming the tube into a series of sinusoidal bends, and then wrapping that pattern around a mandrel, e.g., as described in U.S. Pat. Nos. 5,019,090 and 5,135,536, the entireties of which are incorporated herein by reference.

It further will be appreciated by those skilled in the art that the heat treatment of tube 20 may be performed prior to insertion of therapeutic agent 30 into lumen 22. Similarly, pores 32 may be formed in a lateral surface of tube 20 after the step of heat treating tube 20, and pores 32 optionally may be formed prior to insertion of therapeutic agent 30 into lumen 22.

Referring now to FIGS. 5A-5B, a preferred method of using drug eluting stent 33 of FIG. 4 is described. Stent 33 is provided in a contracted state within delivery sheath 42 whereby tube 20 is constrained in a longitudinally expanded and radially contracted position near a distal end of sheath 42, as shown in FIG. 5A. The distal end of sheath 42 is advanced to a desired site in vessel V under fluoroscopic guidance, preferably using a guidewire (not shown).

Push rod 44 having proximal and distal ends is disposed within sheath 42 and abuts proximal end 21 of tube 20. When sheath 42 is positioned at a desired treatment site, the proximal end of sheath 42 may be retracted by a physician while push rod 44 is held stationary to cause tube 20 to be ejected from the distal end of sheath 42. Tube 20 self-deploys within vessel V to form coil-shaped stent 33, as shown in FIG. 5B. The stent serves to maintain patency in vessel V while blood is permitted to flow through apertures 35.

In accordance with principles of the present invention, therapeutic agent 30 is eluted from pores 32 for an extended period of time after implantation of stent 33 in vessel V, as shown in FIG. 5B. The controlled rate at which agent 30 is eluted may be determined by formulating therapeutic agent 30 with a bioabsorbable polymer (not shown), prior to the step of inserting therapeutic agent 30 into lumen 22. The bioabsorbable polymer mediates the delivery of therapeutic agent 30 to vessel V at a controlled rate after implantation of the stent as a result of the degradation of the polymer by continual blood flow in the vessel.

Alternatively, agent 30 may be formulated to have a highly viscous characteristic. The viscous characteristic is expected to ensure that therapeutic agent 30 is retained within lumen 22 during delivery of the stent, and then eluted from pores 32 in a slow, controlled fashion over an extended period of time. In accordance with principles of the present invention, the elution of therapeutic agent 30 over an extended period of time provides persistent exposure to the therapeutic agent, e.g., to reduce the likelihood of restenosis within vessel V.

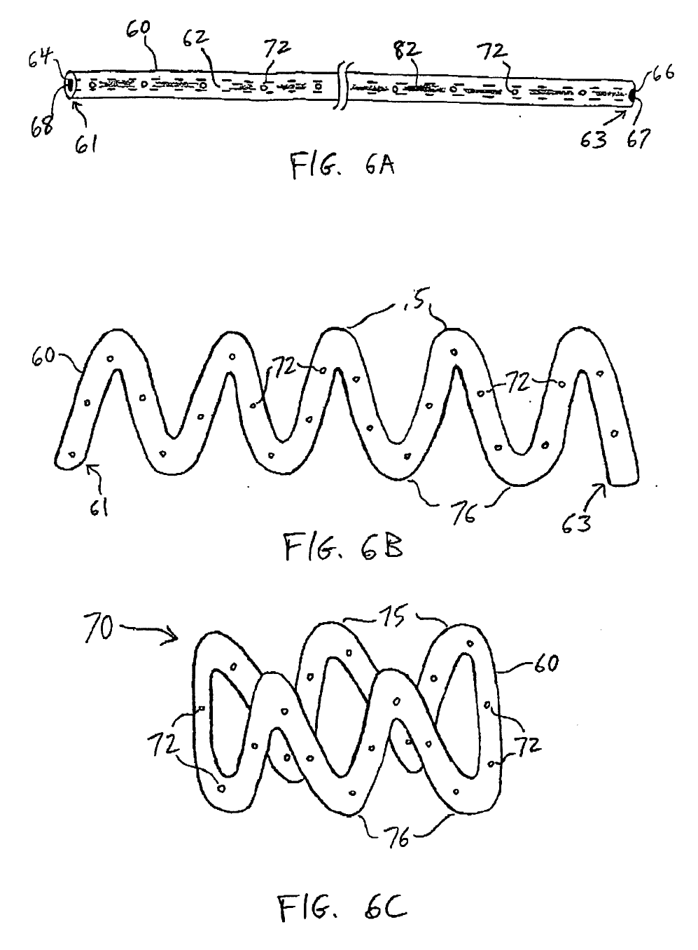

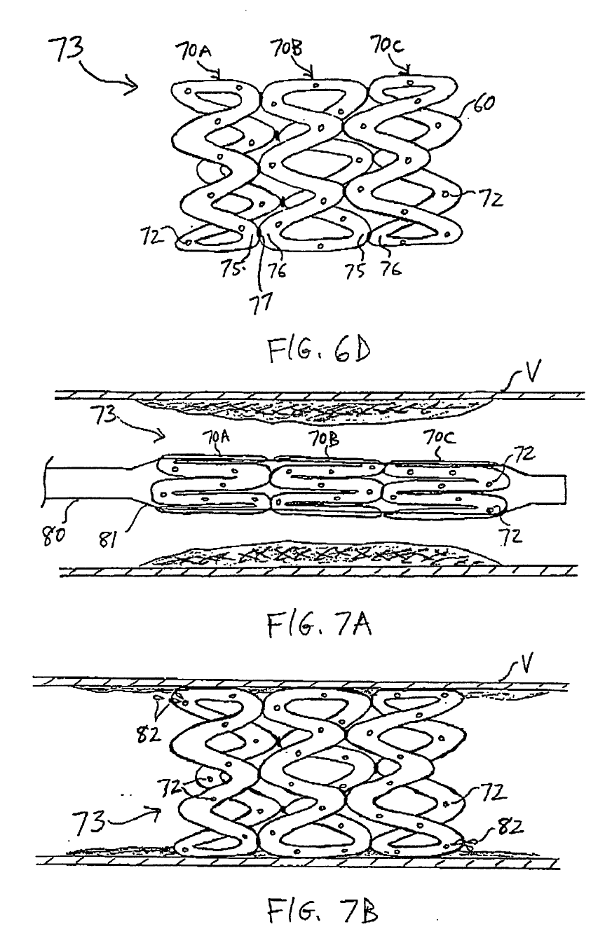

Referring now to FIGS. 6-7, another embodiment of a drug eluting stent constructed in accordance with principles of the present invention is described. In FIG. 6A, tube 60 having proximal and distal ends 61 and 63 and lumen 62 extending therebetween preferably is provided, as described hereinabove with respect to tube 20 of FIGS. 1A-1D. Tube 60 comprises a multiplicity of microscopic pores 72 disposed in a lateral surface of tube 60 and therapeutic agent 82 disposed within lumen 62. Agent 82 may be inserted into lumen 62, e.g., as described hereinabove, and welds 68 and 67 may be used to plug proximal and distal openings 64 and 66, respectively. Tube 60 preferably is fabricated from steel, e.g., stainless steel.

In the embodiment of FIGS. 6, tube 60 is deformed into a configuration having a plurality of upper peaks 75 and lower peaks 76, as shown in FIG. 6B. Tube 60 may be deformed using a die (not shown) that imposes a compressive force upon the tube to cause the desired deformation. Proximal end 61 and distal end 63 then may be joined together, e.g., using a weld, to form circumferential ring 70, as shown in FIG. 6C. Alternatively, ends 61 and 63 may be welded together before the ring is molded into series of peaks and valleys.

In a preferred embodiment, a plurality of circumferential rings 70 are affixed together to form stent 73, as shown in FIG. 6D. As illustrated, stent 73 comprises three circumferential rings 70A-70C, although it will be apparent to those skilled in the art that greater or fewer rings may be used. Lower peaks 75 of circumferential ring 70A preferably are welded to upper peaks 76 of ring 70B at joints 77, as shown in FIG. 6D, while lower peaks 75 of circumferential ring 70B are welded to upper peaks 76 of ring 70C to form stent 73. Alternatively, the rings may be coupled to one another using flexible connectors, as described, e.g., in U.S. Pat. No. 6,068,656, which is incorporated herein by reference.

Referring now to FIGS. 7, a preferred method for using stent 73 of FIG. 6D is described. Circumferential rings 70A-70C of stent 73 preferably are compressed and crimped onto balloon 81 of conventional balloon catheter 80 in a contracted state. Balloon 81 is positioned at a desired location within vessel V under fluoroscopy and inflated to cause radial expansion of stent 73 from the contracted state to a deployed state, as shown in FIG. 7B. Stent 73 serves to maintain patency in vessel V in the deployed state while blood is permitted to flow through circumferential rings 70A-70C. In an alternative embodiment, stent 73 may comprise a shape-memory material, whereby an outer sheath (not shown) may be disposed over stent 73 to confine stent 73 in a contracted state, while retraction of the outer sheath causes stent 73 to self-expand to the deployed shape.

As described hereinabove with respect to FIG. 5B, therapeutic agent 82 is eluted from pores 72, as shown in FIG. 7B, for an extended period of time after implantation of stent 73 in vessel V. Agent 82 preferably is used in conjunction with a bioabsorbable polymer that mediates the delivery of agent 82 to vessel V by biodegrading over an extended period of time.

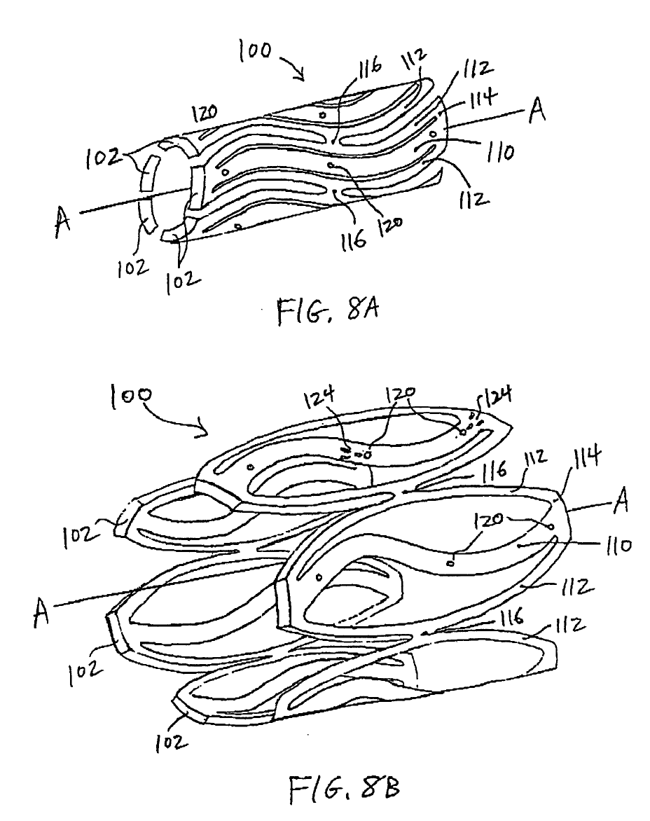

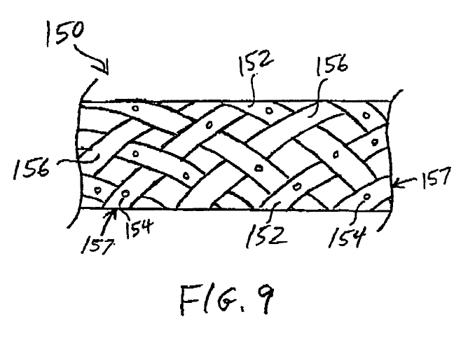

Referring now to FIGS. 8, a further alternative embodiment of a drug eluting stent constructed in accordance with principles of the present invention is described. In FIG. 8A, stent 100 comprises a plurality of unit cells 102 having a “bistable function,” defined herein as only two configurations in which it is stable without the need for an external force to hold it in that shape. The first configuration in which unit cells 102 are stable is a contracted position shown in FIG. 8A, and the second stable configuration is a deployed configuration shown in FIG. 8B.

In a preferred embodiment, each unit cell 102 comprises one first segment 110 that is coupled to two second segments 112 at outer hinges 114, as shown in FIG. 8A. First segments 110 are relatively rigid while second segments 112 are more flexible than first segments 110.

Adjacent unit cells 102 preferably are arranged so that two second segments 112 are disposed between first segments 110, as shown in FIG. 8A. Adjacent second segments 112 preferably are connected by joint 116 that is disposed near a midpoint of second segments 112. In FIG. 8A, the sinusoidal configurations of rigid first segments 110 serve to hold flexible second segments 112 in stable, sinusoidally-shaped contracted states.

In FIG. 8B, stent 100 is shown in a fully deployed state. Unit cells 102 preferably are deployed to the shape shown in FIG. 8B by applying a uniform radially outward force, e.g., by inflating a balloon (not shown), that is sufficient to overcome the resistance of second segments 112 in their stable, sinusoidal-shaped contracted states. Once the force has overcome this resistance, second segments 112 will automatically snap into their respective stable, convex-shaped deployed positions, as shown in FIG. 8B. Second segments 112 provide the radial expansion of stent 100, while first segments 110 substantially maintain their original shapes.

In accordance with principles of the present invention, any of first segments 110 and/or second segments 112 may comprise at least one lumen, whereby the lumen is in fluid communication with a multiplicity of pores 120. As described hereinabove, pores 120 are configured to elute therapeutic agent 124 over an extended period of time after deployment of stent 100 in a patient's vessel. It should be understood by those skilled in the art that multiple therapeutic agents may be provided.

Referring now to FIG. 9, a further alternative embodiment of the present invention is described. Drug eluting stent 150 is a mesh stent that may be configured in accordance with mesh stents that are per se known in the art. Stent 150 preferably comprises a plurality of tubes 152 that are braided in two opposing directions to form the stent, as shown in FIG. 9. In accordance with principles of the present invention, each tube 152 comprises lumen 157 that is in fluid communication with a multiplicity of pores 154. Tubes 152 preferably are manufactured as described hereinabove with respect to tube 20 of FIGS. 1A-1D so that lumens 157 are configured to provide a therapeutic agent that may be eluted from pores 154 after deployment of stent 150 in a patient's vessel. Stent 150 may additionally comprise at least one solid wire segment 156 braided together with tubes 152, as shown in FIG. 9, which may be desirable for structural purposes or to reduce manufacturing costs of the stent.

While preferred illustrative embodiments of the invention are described above, it will be apparent to one skilled in the art that various changes and modifications may be made therein without departing from the invention. The appended claims are intended to cover all such changes and modifications that fall within the true spirit and scope of the invention.

What is claimed is:

1. An implantable device for delivering a therapeutic agent into a vessel, the device comprising:
a stent formed from a tubular member, the tubular member having a lumen and a multiplicity of pores in fluid communication with the lumen; and
a therapeutic agent disposed within the lumen,
wherein the therapeutic agent is configured to be eluted from the lumen into the vessel through the multiplicity of pores after implantation of the stent within the vessel.

2. The device of claim 1 wherein the lumen extends from a proximal end of the tubular member to a distal end of the tubular member.

3. The device of claim 1 wherein the tubular member comprises at least one solid section that segregates the lumen into two or more compartments.

4. The device of claim 3 wherein a compartment is disposed between a first solid section and a second solid section.

5. The device of claim 1 wherein the pores are spaced apart at variable distances with respect to one another.

6. The device of claim 1 wherein the pores are disposed circumferentially about an exterior surface of the tubular member.

7. The device of claim 1 wherein the multiplicity of pores vary in size with respect to one another.

8. The device of claim 1 wherein the multiplicity of pores vary in shape with respect to one another.

9. The device of claim 1 wherein the tubular member comprises a contracted state suitable for insertion into a vessel, and a deployed state in which the tubular member comprises a coil shape configured to contact an inner wall of the vessel.

10. The device of claim 9 wherein the tubular member comprises a shape memory material.

11. The device of claim 1 wherein the tubular member is deformed into a configuration having a plurality of upper peaks and lower peaks, whereby a proximal end of the tubular member is affixed to a distal end of the tubular member to form a circumferential ring.

12. The device of claim 11 wherein a plurality of circumferential rings are affixed together.

13. The device of claim 1 wherein a plurality of the tubular members are braided to form a mesh.

14. The device of claim 13 further comprising at least one solid segment braided together with the plurality of tubular members.

15. A method for manufacturing a stent for use in a vessel, the method comprising:
providing a tube having a lumen;
forming a multiplicity of pores in a lateral surface of the wire and in fluid communication with the lumen;
forming a stent from the tube; and
inserting a therapeutic agent into the lumen,
wherein the therapeutic agent is formulated to be retained within the lumen during delivery of the stent and thereafter eluted within the vessel.

16. The method of claim 15 wherein the therapeutic agent is inserted into a proximal opening of the tube.

17. The method of claim 15 wherein the tube is formed from a shape-memory alloy and forming a stent from the tube comprises processing the tube to deploy to a coil shape.

18. The method of claim 15 wherein forming a stent from the tube further comprises:
deforming the tube into a configuration having a plurality of upper peaks and lower peaks;
affixing a proximal end of the tube to a distal end of the tube to form a circumferential ring; and
affixing a plurality of circumferential rings together to form the stent.

19. The method of claim 15 wherein forming a stent from the tube comprises braiding a plurality of tubes to form a mesh stent.

20. The method of claim 19 further comprising braiding at least one solid wire segment together with the plurality of tubes.

21. The method of claim 15 wherein the pores are disposed circumferentially about an exterior surface of the tube.

22. The method of claim 15 wherein the pores are disposed at variable distances with respect to one another.

23. A method for delivering a therapeutic agent into a vessel, the method comprising:
providing a stent formed from a tubular member, the tubular member having a lumen with a therapeutic agent disposed therein and a multiplicity of pores in fluid communication with the lumen;
implanting the stent within the vessel; and
eluting the therapeutic agent from the lumen into the vessel through the multiplicity of pores.

24. The method of claim 23 further comprising providing a bioabsorbable polymer formulated with the therapeutic agent, wherein the bioabsorbable polymer modulates elution of the therapeutic agent.
